(19) United States
(12) Patent Application Publication
(10) Pub. No.: US 2026/0027081 A1
(43) Pub. Date: Jan. 29, 2026

(54) ARTEMISININ-BASED COMBINATION THERAPY FOR TREATING VIRAL MEDIATED DISEASE

(71) Applicant: KRYPTONITE GROUP, Ltd., Freeport (BS)

(72) Inventors: Robert Lewis Steele, Palm Beach Gardens, FL (US); Anthony Fedele Musso, West Palm Beach, FL (US); David D. Mundschenk, Hollywood, FL (US); Russell Van de Casteele, Pompano Beach, FL (US)

(21) Appl. No.: 19/349,421

(22) Filed: Oct. 3, 2025

Related U.S. Application Data
(63) Continuation of application No. 14/201,749, filed on Mar. 7, 2014, now abandoned.
Application No. 14/201,749 is a continuation-in-part of application No. 13/542,702, filed on Jul. 6, 2012, now abandoned.

(51) Int. Cl. A61K 31/357; A61K 9/00; A61K 31/365; A61K 31/4741; A61K 47/18; A61K 47/44

(52) U.S. Cl. CPC A61K 31/357; A61K 9/006; A61K 31/365; A61K 31/4741; A61K 47/186; A61K 47/44; A61K 9/0019; Y02A 50/30

(57) ABSTRACT

The present invention describes a method of treating individuals suffering from microbial infections, including a viral infection such as Dengue Fever, using an improved Artemisinin Combination Therapy (ACT), known as Tri-ACT. The improved ACT therapy includes administering a combination of three drugs. In one embodiment of the present invention, the method includes administering to an individual a first composition comprising a therapeutically effective amount of an artemether spray sublingually. The individual is then administered a second composition, a therapeutically effective amount of artesunate. A third composition, an effective amount of berberine, or its pharmaceutically acceptable derivatives or salts is then administered to the individual.

### CROSS REFERENCE TO RELATED APPLICATIONS

In accordance with 37 C.F.R. 1.76, a claim of priority is included in an Application Data Sheet filed concurrently herewith. Accordingly, the present invention is a continuation of U.S. application Ser. No. 14/201,749, entitled, “ARTEMISININ-BASED COMBINATION THERAPY FOR TREATING VIRAL MEDIATED DISEASE”, filed Mar. 7, 2014, which claims priority as a Continuation-in-Part of U.S. patent application Ser. No. 13/542,702, entitled, “ARTEMISININ-BASED COMBINATION THERAPY FOR TREATING VIRAL MEDIATED DISEASE”, filed Jul. 6, 2012, which claims priority to U.S. Provisional Patent Application No. 61/878,848, entitled “ARTEMETHER SPRAY”, filed Sep. 17, 2013. The contents of the above referenced applications are incorporated herein by reference in its entirety.

### FIELD OF THE INVENTION

The present invention relates to microbial therapies, and particularly to a method of treating individuals suffering from a viral related disease using an improved Artemisinin based therapy, and more particularly to a method of treating an individual suffering from Dengue using an improved Artemisinin Combination Therapy (ACT).

### BACKGROUND OF THE INVENTION

Dengue is a viral mediated disease spread by several species of mosquitoes within the Aedes species, most commonly by Aedes aegypti. Dengue is an acute multi-systemic viral infection that is so dynamic that within a remarkably short period of time, a mild case can be transformed into a severe one attributed to increasing capillary permeability/fragility leading to increase plasma leakage, hypotension, shock and death if left untreated. According to the World Health Organization (WHO), dengue is the most rapidly spreading mosquito-borne viral disease in the world. In the last 50 years, the incidence has increased 30-fold with an increasing geographic expansion to new countries. It is caused by one of four similar RNA viruses of the family Flaviviridae, DEN-1, DEN-2, Den-3, and DEN-4. All four serotypes can result in full blown Dengue. Infection with a serotype of Dengue may produce a spectrum of clinical disease from non-specific viral syndrome to severe fatal hemorrhagic disease. Symptoms of Dengue include sudden high fever, often in the range of 104-105 degrees Fahrenheit, about 4-7 days after the bite. A characteristic body rash, similar to the skin rash seen in measles, appears several days after the fever begins. In some cases, the disease progresses to dengue hemorrhagic fever, resulting in bleeding, low blood platelet levels, plasma leakage, and dengue shock syndrome.

Dengue is the second most important infectious tropical disease after malaria, as more than one-third of the world's population live in areas at risk of transmission. There are estimated to be 200 million individuals infected with the Dengue virus every year, with 10,000,000 patients hospitalized for hemorrhagic dengue fever, with about 5% of such cases resulting in death. Dengue virus infections are endemic in more than 100 tropical countries, including countries in Asia, the Pacific, Africa, Latin America and the Caribbean. While such places tend to have unplanned and uncontrolled urbanization, as well as high poverty, an increase in air travel, lack of effective mosquito control, and military deployment into risk regions can result in outbreaks in more developed countries. In 2009, Dengue infection was reported in the United States by a Florida resident who had not travelled outside the U.S.

There are no known treatments or vaccines to prevent infection with Dengue virus, In fact, the most effective measure to prevent the disease is to avoid mosquito bites. While protective clothing and mosquito irradiation programs theoretically provide protection, what is needed in the art is an easy to administer method of treating individuals suffering Dengue.

### SUMMARY OF THE INVENTION

The present invention describes a therapy for individuals suffering from a viral infection based on Artemisinin Combination Therapy (ACT). In contrast to most ACT therapies which utilize a combination dual drug therapy, the present invention describes a method which uses a combination of three drugs, In one embodiment of the present invention, the method includes administering to an individual a first composition. The first composition comprises of therapeutically effective amount of an artemisinin derivate or its salt, such as an artemether or artemether spray delivered sublingually. The individual is then administered a second composition. The second composition comprises of a therapeutically effective amount of second artemisinin derivate or its' salt. The second artemisinin derivate differs from the first composition and is preferably artesunate. A third anti-microbial composition is administered to the individual. The third composition comprises an effective amount of berberine, or its pharmaceutically acceptable derivatives or salts. The second and third compositions are administered to the individual for additional periods, such as for two or three days.

In an alternative embodiment, the treatment is administered to an individual suffering from disease transmitted by an arthropod, such as a mosquito or tick,

In an alternative embodiment, the treatment is administered to an individual suffering from a disease mediated by a virus of the Flaviviridae family of viruses.

In an alternative embodiment, the treatment is administered to an individual suffering from a disease mediated by a virus similar to viruses in the Flaviviridae family of viruses.

In an alternative embodiment, the treatment is administered to an individual suffering from a disease mediated by a virus causing hemorrhagic fever.

In an alternative embodiment, the treatment is administered to an individual suffering from a disease mediated by a Dengue virus.

In an alternative embodiment, the treatment is administered to an individual suffering from a disease mediated by a virus causing Dengue fever.

When used for individuals suffering from Dengue, the TriAct therapy was proven safe and effective, with no deaths or adverse reactions. The TriAct therapy treatment shortened the disease process, decreased viral antigen (NS1), and decreased the risk reduction of bleeding. The TriAct therapy resulted in faster normalization of the hematological picture and early resolution of the clinical symptoms. Individuals taking the TriAct therapy, when compared to a control group, were shown to have an enhancement of the immune system allowing early production of neutralizing antibodies.

The present invention further describes an improved artemether based supplement and/or pharmaceutical composition which is administered as a sublingual spray. The supplement and/or pharmaceutical composition contains an Artemisinin derivative, an allergenic minimizing carrier, and a mucosal absorption enhancer. The present invention further relates to a method of manufacturing the artemether based supplement and/or pharmaceutical composition and the uses thereof for delivery to an individual in need thereof.

As used herein, the term “Flaviviridae family of viruses” includes about 70 members which have linear, single-stranded RNA genomes of positive polarity. The family includes the Genus Flavivirus, Genus Hepacivirus, Genus Hepatitis G Virus, and Genus Pestivirus. Major diseases caused by the Flaviviridae family include: Dengue, Japanese encephalitis, Kyasanur Forest disease, Murray Valley encephalitis, St. Louis encephalitis, Tick-borne encephalitis, West Nile encephalitis, Yellow fever, and Hepatitis C Virus Infection.

As used herein, the term “pharmaceutically acceptable excipient” or “pharmaceutically acceptable carrier,” generally refers to organic or inorganic materials, non-toxic, inert solid, semi-solid or liquid filler, diluent, encapsulating material or formulation auxiliary of any type which cannot react with active ingredients, Excipients include but are not limited to sugars, such as lactose, glucose and sucrose; starches such as corn starch and potato starch; cellulose and its derivatives such as sodium carboxymethylcellulose, ethylcellulose, and cellulose acetate; powered tragacanth; malt; gelatin; talc; stearic acids; magnesium stearate; calcium sulfate; cocoa butter and suppository waxes; vegetable oils, such as peanut oil, cotton seed oil, sesame oil, olive oil, corn oil and oil of theobroma; esters such as but not limited to ethyl oleate and ethyl laurate; polyols such as propylene glycol, glycerine, sorbitol, mannitol, and polyethylene glycol; agar; alginic acids; buffering agents such as but not limited to, magnesium hydroxide and aluminum hydroxide; pyrogen-free water; isotonic saline; and phosphate buffer solution; skim milk powder; as well as other non-toxic compatible substances used in pharmaceutical formulations. Wetting agents and lubricants such as sodium lauryl sulfate and magnesium stearate, as well as coloring agents, flavoring agents, sweetening agents, lubricants, releasing agents, perfuming agents, carriers, tabletting agents, stabilizers, antioxidants and preservatives can also be present.

As use herein, “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “therapeutically effective amount” generally refers to an amount of an agent, for example the amount of a compound as an active ingredient, that is sufficient to effect treatment as defined herein when administered to an individual, such as a mammal, preferably a human in need of such treatment. A therapeutically effective amount of a compound, salt, analog, or derivative of the present invention will depend on a number of factors including, for example, the age and weight of the subject, the precise condition requiring treatment and its severity, the nature of the formulation, and the route of administration, and will ultimately be at the discretion of the attendant physician.

As used herein, the term “treat”, “treating” or “treatment” refers to the administration of therapy to a subject, particularly a mammal, more particularly a human, who already manifests at least one symptom of a disease to obtain a desired pharmacological and physiological effect. Such subject includes an individual who is diagnosed as having a disease. The term may also include preventing the disease, i.e. causing the clinical symptoms of the disease not to develop in a mammal that may be exposed to or predisposed to the disease but does not yet experience or display symptoms of the disease, inhibiting the disease, i.e., arresting or reducing the development of the disease or its clinical symptoms, or relieving the disease, i.e., causing regression of the disease or its clinical symptoms.

As used herein, “virus causing hemorrhagic fever” describes RNA viruses such as those of Filoviridae, Arenaviridae, Bunyaviridae or Flaviviridae family, include but are not limited to ebola viruses, marburg viruses, flexal viruses, guanarito viruses, jumin viruses, lassa fever viruses, machupo viruses, sabia viruses, crimea-congo hemorrhagic fever viruses, rift valley fever viruses, hantaan viruses, dengue viruses, kyasanur forest disease viruses, omsk hemorrhagic fever viruses, and yellow fever viruses. In one aspect of the invention, the Hemorrhagic fever viruses are dengue viruses.

Accordingly, it is an objective of the present invention to provide an improved Artemisinin Combination Therapy to individuals in need thereof.

It is a further objective of the present invention to provide an improved Artemisinin-based Combination Therapy to individuals suffering from a viral disease.

It is a further objective of the present invention to provide an improved Artemisinin-based Combination Therapy to individuals suffering from a viral disease mediated by arthropods, such as a mosquito.

It is a still further objective of the present invention to provide an improved Artemisinin-based Combination Therapy to individuals suffering from a viral disease mediated by a virus of the family Flaviviridae.

It is a further objective of the present invention to provide an improved Artemisinin-based Combination Therapy incorporating a sublingual delivery step to individuals suffering from a viral disease.

It is yet another objective of the present invention to provide an improved Artemisinin-based Combination Therapy incorporating sublingual delivery step to individuals suffering from a viral disease mediated by a virus of the family Flaviviridae.

It is a still further objective of the present invention to provide an improved Artemisinin-based Combination Therapy incorporating delivery of an Artemisinin derivative using a sublingual, spray-based delivery route of administration to individuals suffering from Dengue.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from a viral disease.

It is a still further objective of the present invention to provide an improved artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from a disease mediated by an arthropod.

It is a further objective of the present invention to provide an improved Artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from Dengue.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from a disease mediated by an arthropod.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from Dengue which is safe and effective.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from Dengue which shortens the disease process.

It is yet another objective of the present invention to provide an improved Artemisinin-based Combination Therapy incorporating delivery of an Artemisinin derivative using sublingual, spray-based delivery route of administration in combination with oral delivery of a second, independent artemisinin derivative and a berberine, or its derivatives, for individuals suffering from Dengue which decreases NS1 viral antigen.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from Dengue which decreases the risk reduction of bleeding.

It is yet another objective of the present invention to provide an improved Artemisinin-based Combination Therapy incorporating delivery of an Artemisinin derivative using a sublingual, spray-based delivery route of administration in combination with oral delivery of a second, independent artemisinin derivative and a berberine, or its derivatives, for individuals suffering from Dengue which results in faster normalization of the hematological picture and early resolution of the clinical symptoms

It is yet another objective of the present invention to provide an improved Artemisinin-based Combination Therapy incorporating delivery of an Artemisinin derivative using sublingual, spray-based delivery route of administration in combination with oral delivery of a second, independent artemisinin derivative and a berberine, or its derivatives, for individuals suffering from Dengue which results in enhancement of the immune system allowing early production of neutralizing antibodies.

It is an objective of the present invention to provide an improved artemether based supplement and/or pharmaceutical composition for treating individuals suffering from disease

It is a still further objective of the present invention to provide an improved artemisinin-based Combination Therapy incorporating delivery of an artemisinin derivative using a sublingual, spray-based delivery route of administration in combination with oral delivery of a second, independent artemisinin derivative and a berberine, or its derivatives, for individuals suffering from a disease in which the artemether based supplement and/or pharmaceutical composition can be delivered as a sublingual spray for treating individuals suffering from disease and contains a carrier that minimizes the risk of an allergic response,

It is yet another objective of the present invention to provide an artemether supplement and/or pharmaceutical composition which can be delivered as a sublingual spray for treating individuals suffering from disease and contains carrier that enhances mucosal absorption.

It is a still further objective of the invention to provide an artemether supplement and/or pharmaceutical composition which can be delivered as a sublingual spray for treating individuals suffering from disease and contains a carrier that minimizes the risk of an allergic response and enhances mucosal absorption.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a graph comparing individuals diagnosed with Dengue and treated with the TriAct therapy versus individuals diagnosed with Dengue and treated using standard care protocol with respect to fever longevity;

FIG. 2 is a graph comparing individuals diagnosed with Dengue and treated with the TriAct therapy versus individuals diagnosed with Dengue and treated using standard care protocol with respect to the number of days suffering with abdominal pain;

FIG. 3 is a graph comparing individuals diagnosed with Dengue and treated with the TriAct therapy versus individuals diagnosed with Dengue and treated using standard care protocol with respect to the number of days suffering with abdominal tenderness;

FIG. 4 is a graph comparing individuals diagnosed with Dengue and treated with the TriAct therapy versus individuals diagnosed with Dengue and treated using standard care protocol with respect to the number of days before testing negative for NS1 antigen;

FIG. 5 is a graph comparing individuals diagnosed with Dengue and treated with the TriAct therapy versus individuals diagnosed with Dengue and treated using standard care protocol with respect to the number of days to normalize absolute platelet counts;

FIG. 6 is a graph comparing individuals diagnosed with Dengue and treated with the TriAct therapy versus individuals diagnosed with Dengue and treated using standard care protocol with respect to the number of days to become positive for IgM;

FIG. 7 is a graph comparing individuals diagnosed with Dengue and treated with the TriAct therapy versus individuals diagnosed with Dengue and treated using standard care protocol with respect to the number of days to become positive for IgG.

### DETAILED DESCRIPTION OF THE INVENTION

While the present invention is susceptible of embodiment in various forms, there is demonstrated and will hereinafter be described a presently preferred, albeit not limiting, embodiment with the understanding that the present disclosure is to be considered an exemplification of the present invention and is not intended to limit the invention to the specific embodiments illustrated.

The present invention describes a novel combination therapy for the treatment of microbial infections, such as viral infections. As an illustrative example, the present invention has been found to be useful in treating diseases mediated by arthropods and/or a viral infection from the Flaviviridae family, such as the viruses causing Dengue. The combination therapy in accordance with the present invention is based on Artemisinin Combination Therapy (ACT). The present invention uses a tri-therapy modality, using a unique combination of three compositions: artemether, artesunate, and berberine, The novel combination therapy may be referred to generally as TriAct therapy, for the triple components that make up combination therapy.

### Artemisinin and Artemisinin Derivatives

Artemisinin, having an IUPAC name of 3R,5aS,6R,8aS,9R,12S,12aR)-octahydro-3,6,9-trimethyl-3,12-epoxy-12H-pyrano[4,3-j]-1,2-benzodioxepin-10(3H)-one, and also known as Qinhaosu, is a natural product derived from the Chinese herb Artemisia annua known to have anti-viral activities, see Efferth et al. The Antiviral Activities of Artemisinin and Artesunate. Clin. Inf. Dis. 2008:47:804-11. It has a chemical structure of:

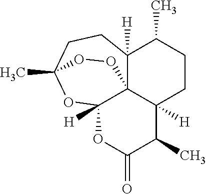

Artemisinin is a sesquiterpene lactone containing an unusual peroxide bridge. This peroxide is believed to be responsible for the drug's mechanism of action. Few other natural compounds with such a peroxide bridge are known; see Artemisinin and a new generation of antimalarial drugs”. Education in Chemistry. July 2006. http://www.rsc.org/Education/EiC/issues/2006July/Artemisinin.asp. It is widely used for the treatment of malaria, particularly in combination with other drugs such as mefloquine (ASMQ), lumefantrine (such as sold under the trade name Coartem), amodiaquine (such as sold under the trade names Camoquin, Flavoquine, ASAQ), piperaquine (such as sold under the trade name Duo-Cotecxin), Artemisinin and pyronaridine (such as sold under the trade name Pyramax). Artemisinin derivatives are typically used as prodrugs of the biologically active metabolite dihydroartemisinin, which is active during the stage when the parasite is located inside red blood cells. The mechanism through which artemisinin derivatives kill the parasite is believed to act via perturbing redox homeostasis in malaria parasites. Accordingly, such compositions may include free-radical production in the parasite food vacuole and inhibition of a parasite calcium ATPase. A key advantage of artemisinins is rapid action against all of the erythrocytic stages of the parasite, including transmissible gametocytes, resulting in a rapid clinical benefit and decreased transmission of malaria; see Rosenthal, Artesunate for Treatment of Severe Falciparum Malaria, N. Engl J Med 2008, 358:1829-1836.

One of the disadvantages of artemisinin is its poor physical properties resulting in poor bioavailability, limiting its effectiveness. For example, the compound is sparingly soluble in either water or oils and thus not readily absorbable by the gastrointestinal tract. As a result, numerous semi-synthetic derivatives have been developed, including artesunate (water-soluble: for oral, rectal, intramuscular, or intravenous use), artemether (lipid-soluble: for oral, rectal ox intramuscular use) dihydroartemisinin, artelinic acid, artenimol and artemotil. As used in the present invention, artemisinin derivatives include, but are not limited to artesunate, dihydroartemisinin, dihydroartemisinin hemisuccinate, dihydrodroartemisinin succinate, sodium artesunate, stabilized forms of artesunate, stabilized forms of sodium artesunate, dihydroartemisitene dimers described in U.S. Pat. No. 7,098,242, amino-functionalized 1,2,4-trioxanes as described in U.S. Pat. No. 7,071,226, artemisinin endoperoxides as described in U.S. Pat. No. 6,984,640, spiro and dispiro 1,2,4-trioxolane antimalarials as described in U.S. Pat. No. 6,906,205, mixed steroidal 1,2,4,5-tetraoxane compounds as described in U.S. Pat. No. 6,906,098, arteether as described in U.S. Pat. No. 6,750,356, substituted 1,2, 4-trioxanes as described in U.S. U.S. Pat. No. 6,737,438, Artemisia annua extracts as described in U.S. Pat. No. 6,685,972, artemether as described in U.S. Pat. No. 6,683,193, trioxane derivatives based on artemisinin as described in U.S. Pat. No. 6,649,647, trioxane dimer compounds as described in U.S. RE38,117, conjugates of artelinic acid as described in U.S. Pat. No. 6,461,603, arteethers from dihydroartemisinin as described in U.S. Pat. No. 6,346,631, artemisinine or artemisinene derivatives as described in U.S. Pat. No. 6,306,896, C-10 carbon substituted artemisinin-like trioxane compounds as described in U.S. Pat. No. 6,160,004, water-soluble trioxanes as described in U.S. Pat. No. 6,136,847, alpha arteether as described in U.S. Pat. No. 6,127,405, artemisinin dimers as described in U.S. Pat. No. 5,856,351, (+)-deoxoarteminisinin and analogs of (+)-deoxoartemisinin as described in U.S. Pat. No. 5,225,562, and 10-substituted ether derivatives of dihydroartemisinin as described in U.S. Pat. No. 5,225,427, as well as its salts or other derivatives thereof as known to one of skill in the art.

Artemether is a methyl-ether derivative of dihydroartemisinin derived from artemisinin. It has an IUPAC chemical name of (+)-(3-alpha,5a-beta,6-beta,8a-beta,9-alpha,12-beta,12aR)-decahydro-10-methoxy-3,6,9-trimethyl-3,12-epoxy-12H-pyrano(4,3-j)-1,2- benzodioxepin, and having a chemical formula:

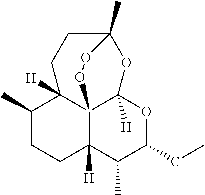

Artemether is known to be effective against blood schizots of several malaria causing parasites, and is used as an ACT drug.

Artesunate, also known as dihydroartemisinin hemisuccinate and its salts, has a IUPAUC chemical name of 3R,5as,6R,8aS,9R,10S,12R,12aR)-Decahydro-3,6,9-trimethyl-3,12-epoxy-12H-pyrano(4,3-j)-1,2benzodioxepin-10-ol hydrogen succinate, with a chemical formula of:

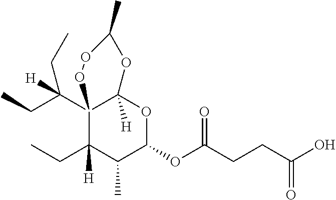

Artesunate is used primarily as a treatment for malaria.

Dosage for Artemisinin, Artemisinin salts and derivatives.

The oral dosage range for Artemisinin, Artemisinin salts and derivatives is between about 1 mg to about 1,500 mg per dose administered to three times daily. More preferably, the oral dosage range for artemisinin, artemisinin salts and derivatives is between about 20 mg to about 250 mg per dose if taken one to three times daily. Most preferably, the oral dosage ranges for artemisinin, artemisinin salts and derivatives is between about 40 mg to about 100 mg per dose taken one to three times daily.

Berberine, salts thereof, and Berberine derivatives.

Berberine is a quaternary ammonium salt from the protoberberine group of isoquinoline alkaloids. It has a IUPAC name of (5,6-Dihydro-9,10 dimethoxybenzo[g]-1,3-benzodioxolo[5,6-a]quinolizinium), with a chemical formula of:

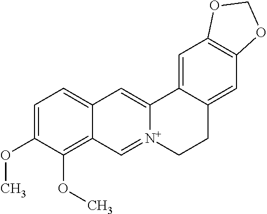 [SMILES: COc1ccc2cc3[n+](cc2c1OC)CCc1cc2c(cc1-3)OCO2]

It is found in various plant species of Berberis (e.g. Berberis aquifolium (Oregon grape), Berberis vulgaris (Barberry), and Berberis aristata (Tree Turmeric)), as well as other plant families, including but not limited to Hydrastis canadensis (Goldenseal), Phellodendron amurense (Amur Cork Tree, Huang Bai, Huang Po, Po Mu) and Coptis chinensis (Chinese Goldthread, Huang-Lian, Huang-Lien), and Tinospora cordifolia, and to a smaller extent in Argemone mexicana (Prickly Poppy) and Eschscholzia californica (Californian Poppy), Rhizoma coptidis Huanglian Jiedu decoction, San-Huang-Xie-Xin-Tang, Xietianwu, Gegen Quinlian, and Shizhu. Berberine is primarily isolated from the roots, rhizomes, stems, and bark. Berberine has been used for medical use in both Ayurvedic and Chinese medicines, and has recently been tested for anti-bacterial activity and use in diarrhea, prostate cancer and diabetes. It is generally considered to be non-toxic and shows no genotoxic activity.

In accordance with the present invention, berberine, berberine derivatives, or its salts may include, but are not limited to, berberine alkaloid, berberine base, berberinehydrochloride, berberine, berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine, tetrahydroprotoberberine 8-cyanodihydroberberine, dimeric protoberberine alkaloids, demethylated protoberberine alkaloids, quataternary protoberberine alkaloids, protoberberine and protoberberine alkaloids, the salts of berberine, including berberine hydrochloride, berberine chloride, berberine sulfate, berberine tannate and other salts known to one of skill in the art.

### Dosage Range for Berberine, its Salts and Derivatives

The oral dosage range of Berberine is from about 50 mg to about 1,500 mg administered in a single dose two to three times daily, not to exceed 4,500 mg per day. More preferably, the dose of berberine is about 100 mg to about 1,000 mg in a single human dose not to exceed 3,000 mg per day, delivered one to three times daily. The most preferable dosage range for a single dose of berberine is about 200 mg to about 500 mg taken one to three times daily.

The present invention is further described by the following non limiting examples. The following examples illustrate embodiments, albeit preferred embodiments, of routes of administration and forms of the composition. While the preferred forms and/or routes are described, other forms, such as but not limited to tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or formulations aerosolized for inhalation, compositions and formulations for intravesical administration. The compositions may also be developed for inhalational, oral, rectal, vaginal, parenteral, topical, transdermal, pulmonary, intranasal, buccal, ophthalmic, intrathecal, intravenous or any route of administration.

The dosage form of the present invention may include either immediate or controlled release forms. Each composition can be formulated and manufactured by procedures known to one of skill in the art, and may include 100% composition or a mixture of one or more pharmaceutically acceptable excipients or pharmaceutically acceptable carriers. For use of sublingual sprays, liquid artemether can be prepared as a spray form using, for example, the microfluidization process as described in U.S. Pat. No. 6,861,066.

The present invention further describes an alternative sublingual spray, artemether based supplement and/or pharmaceutical composition, a method of manufacturing the improved artemether based supplement and/or pharmaceutical composition, and the use thereof for delivery to an individual in need thereof. The supplement and/or pharmaceutical composition contains an active compound such as an Artemisinin derivative, a pharmaceutically non-allergenic acceptable carrier which reduces the risk of an allergic reaction when administered to an individual, and a mucosal absorption enhancer. In a preferred embodiment, the sublingual spray supplement and/or pharmaceutical composition includes artemether, a pharmaceutically non-allergenic acceptable carrier, such as neutral oil, including a neutral oil such as olive oil (obtained from the fruit of Olea europaea), and a quaternary ammonium salt such as Benzalkonium Chloride which acts as a mucosal absorption enhancer.

The present invention includes an improved artemether supplement and/or pharmaceutical composition utilizing a carrier which reduces the risk of an allergic reaction when administered to an individual susceptible to food based allergies. While Artemisinin and Artemisinin derivatives for use in the treatment of disease is known in the art, such compositions are prepared using arachis (peanut) oil. The use of such oil presents a problem for any supplement or pharmacological routes of administration that require delivery within the oral cavity, such as the mouth whereby the active ingredient is required to diffuse into the circulatory system through tissues under the tongue. As a common trigger to those individuals suffering food induced allergic reactions, using peanut oil as a carrier for sublingual sprays increases the risk of severe injury and even fatalities.

To minimize the risk of potential allergic reactions, the supplement and/or pharmaceutical composition in accordance with the present invention uses olive oil as a carrier for the Artemether as there is no known cases of hypersensitivity to olive oil when taken by mouth.

### Mucosal Absorption Enhancer:

Quaternary ammonium salts may be used to form the supplement and/or pharmaceutical composition. Such quaternary ammonium salts include those commonly used and considered as safe for human use. Such compounds are typically tetrasubstituted ammonium salts in which the substituent groups are preferably hydrocarbon compounds attached to the nitrogen by an N—C bond, and selected from the group consisting of substituted and unsubstituted, saturated and unsaturated, aliphatic and aromatic, and branched and normal chain groups. In all cases the nitrogen atom is pentavalent and is in the positively charged portion of the molecule, thus quaternary ammonium salts are cationic electrolytes.

Preferably, the mucosal absorption enhancer of this invention is benzalkonium chloride, also known alkyl dimethylbenzyl ammonium chloride, alkyldimethyl (phenylmethyl), Quaternary Ammonium Chloride, Ammonyx, and Roccal, Benzalkonium chloride has a chemical formula of:

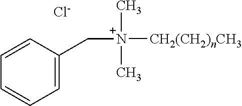

Benzalkonium chloride is commercially available in suitable form from a number of sources, including Sigma Aldrich Chemical Co. Benzalkonium chloride used in an amount effective to serve as a membrane enhance as well as a preservative. The Benzalkonium chloride may be used at a final concentration of between about 0.001% and about 0.1%, by weight, and preferably between about 0.005% and about 0.05%, based on the weight of the composition, and more preferably at or about 0.007% based on the weight of the composition.

Tables 1-5 illustrate examples of the spray artemether supplement and/or pharmaceutical composition in accordance with the present invention.

TABLE 1
Sublingual Spray

| Component | Concentration |
|---|---|
| Artemether | Between about 5.0 mg and 80.0 |
| | mg by weight |
| Acceptable Carrier | As needed |
| Membrane Enhancer | 0.001%-0.05% by weight |
| Preservative | 0.001%-0.05% by weight |

TABLE 2
Sublingual Spray

| Component | Concentration |
|---|---|
| Artemether | Between about 5.0 mg and 80.0 |
| | mg by weight |
| Non allergenic Acceptable | As needed |
| Carrier | |
| Membrane Enhancer | 0.001%-0.05% by weight |
| Preservative | 0.001%-0.05% by weight |

TABLE 3
Sublingual Spray

| Component | Concentration |
|---|---|
| Artemether | Between about 5.0 mg and 80.0 |
| | mg by weight |
| Non-allergenic Acceptable | As needed |
| Carrier: Olive Oil | |
| Quaternary ammonium salts | 0.001%-0.05% by weight |

TABLE 4
Sublingual Spray

| Component | Concentration |
|---|---|
| Artemether | Between about 30.0 mg and |
| | about 80.0 mg by weight |
| Non-allergenic Acceptable | As needed |
| Carrier: Olive Oil | |
| Benzalkonium Chloride | 0.001%-0.05% by weight |

TABLE 5
Sublingual Spray

| Component | Concentration |
|---|---|
| Artemether | About 80.0 mg by weight |
| Non-allergenic Acceptable | As needed |
| Carrier: Olive Oil | |
| Benzalkonium Chloride | 0.007% by weight |

### EXAMPLE

### Method of Preparing Sublingual Artemether Spray, Single dose 80 mg

To prepare the artemether sublingual spray, powdered artemether was obtained. In preparing single spray dosage, 700 mL of extra virgin olive oil was heated to 130 degree in a one liter beaker. The olive oil was cooled to 70 degrees. Once at 70 degrees, 80 grams of the artemether powder was added. The 80 grams of the artemether powder was slowly added to the cooled olive oil under continuous stirring until the powder was dissolved, forming a clear solution. To the artemether in solution, 0.7 ml of 10% Benzalkonium Chloride was added under continuous stirring. The artemether solution was allowed to cool to room temperature. Concentration of artemether was validated using HPLC.

Preferably, the artemether solution described above is packaged in a single dose delivery system. such as an atomizing spray pump as described in U.S. Pat. No. 6,126,038, where the patient can simply spray the composition to the desired place, i.e. under the tongue, until the bottle is completely discharged.

### Methods of Treating Individuals

### Example 1

Method of treating an individual suffering from a disease mediated by a virus of the family Flaviviridae.

A method of treating an individual suffering from a disease mediated by a virus of the family Flaviviridae, comprises the steps of (1) administering to a mammal, preferably a human, a first composition, the first composition comprising a therapeutically effective amount of an artemisinin derivate, preferably artemether at 50-80 mg per dose, preferably 60 mg; (2) administering to the mammal a second composition, the second composition comprising a therapeutically effective amount of a second artemisinin derivate, the second artemisinin derivate differing from the first composition, and preferably being artesunate or its pharmaceutically acceptable derivatives at 1 mg to 1,500 mg per dose, preferably about 20 mg to about 250 mg per dose, three times daily, and most preferably about 40 mg to about 100 mg per dose three times daily; and (3) administering to a mammal a third composition, the third composition comprising a therapeutically effective amount of berberine, or its pharmaceutically acceptable derivatives at about 1 mg to about 1,500 mg per dose, particularly about 100 mg to about 1,000 mg per dose three times a day, and more specifically about 200 mg to about 400 mg per dose, two to three times a day.

The following describes an illustrative, albeit preferred delivery treatment schedule:
Day 1: Delivery of Artemether-single dose.
Delivery of Artesunate and berberine tablets, dosage according to user age and/or weight every eight hours.
Day 2: Delivery of Artesunate and berberine tablets, dosage according to user age and/or weight every eight hours.
Day 3: Delivery of Artesunate and berberine tablets, dosage according to user age and/or weight every eight hours.

### Example 2

Treatment for adults over 165 pounds (75 Kg) suffering from a viral infection using sublingual delivery of an artemether solution: Dengue.

A method of treating an individual suffering from Dengue comprises the steps of (1) administering to an adult mammal, preferably a human, a first composition, the first composition comprising a therapeutically effective amount of artemether; (2) administering to the adult mammal second composition, the second composition comprising a therapeutically effective amount of an artesunate or its pharmaceutically acceptable derivatives; and (3) administering to the adult mammal a third composition, the third composition comprising a therapeutically effective amount of berberine, or its pharmaceutically acceptable derivatives.

An adult human, over 165 pounds (75 Kg), begins the method in accordance with the present invention by administering the first loading dose of the artemether. The artemether is delivered to the individual using preferably, a single dose sublingual spray bottle containing preferably 60 mg of an artemether solution per bottle. Use of a spray offers advantages over injectable form by providing a method of delivery that is easy to administer, does not require a trained medical professional such as a doctor or nurse, and avoids the problems associated with needles, such as fear of spreading disease such as HIV or Hepatitis C and proper disposal. In addition, sublingual drug delivery, particularly sprays, allows the active ingredients improved absorption and enhanced bioavailability.

In use, the user simply removes the bottle from the transportation/delivery packaging and displaces any safety features, such as any tamper proofing outside wrapping, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes, the user administers the second composition comprising a dosage of artesunate, such as 2×50 mg tablets, 3 doses per day (300 mg per day), or every eight hours for 2 days, The user administers the third composition comprising a dosage of berberine, such as 2×400 mg tablets, 3 doses per day (2,400 mg), or every eight hours. Alternatively, the second and third compositions can be administered simultaneously with the first composition.

### Example 3

Treatment for adult humans 66 (30 kg)-165 pounds (75 Kg) suffering from a viral infection using sublingual delivery of an artemether solution: Dengue.

An adult human weighing 66 (30 kg)-165 pounds (75 Kg) begins the method in accordance with the present invention by administering the first loading dose of the artemether. The artemether is delivered to the individual using preferably a single dose sublingual spray bottle containing preferably 60 mg artemether solution per bottle. The user simply removes the bottle from the transportation/delivery packaging and displaces any safety features, such as outside wrapping to indicate tampering, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for predetermined time period, such as 1 minute to 1 hour, and more preferably 30 minutes, the user administers the second composition comprising a dosage of artesunate, such as 1×50 mg tablets, 3 doses per day (150 mg), or every eight hours for 2 days. The user administers the third composition comprising a dosage of berberine, such as 1×400 mg tablet, 3 doses per day (1200 mg), or every eight hours. Alternatively, the second and third compositions can be administered simultaneously with the first composition.

### Example 4

Treatment for human a child 33 pounds (15 kg)-66 pounds (30 Kg) suffering a viral infection using sublingual delivery of an artemether solution: Dengue.

For children or individuals in the range of between 33 pounds (15 kg)-66 pounds (30 Kg), the treatment is the same as described in Example 3.

### Example 5

Treatment for infants and toddlers 33 pounds up to 3 years old (under 15 kg) suffering viral infection using sublingual delivery of an artemether solution: Dengue.

For infants and toddlers 33 pounds up to 3 years old (under 15 kg), the treatment is similar to Example 1. The dosage delivered to the user includes artemether delivered to the individual using preferably a single dose, sublingual spray containing preferably 60 mg artemether solution per bottle, 25 mg artesunate per dosage, 3 doses per day (75 mg total per day), or every eight hours for 2 days, and 200 mg berberine per dosage, 3 doses per day (600 mg total per day), or every eight hours. For infants and toddlers, the tablets can be ground up and mixed with liquids that allow delivery into the users, such as milk, juice, and syrups.

Example 6

Treatment for individuals suffering viral infection using injectable delivery of artemether solution: Dengue.

A method of treating an individual suffering from Dengue comprises the steps of (1) administering to a mammal, preferably a human, a first composition, the first composition comprising a therapeutically effective amount of artemether; (2) administering to the mammal a second composition, the second composition comprising a therapeutically effective amount of artesunate or its pharmaceutically acceptable derivatives; and (3) administering to the mammal a third composition, the third composition comprising a therapeutically effective amount of berberine, or its pharmaceutically acceptable derivatives.

In a preferred embodiment, the method of treatment can be facilitated by the use of a kit comprising an injectable artemether solution as a single dosage unit contained in an ampoule, injection vial, or as an individual single, preloaded injection syringe with needle, a first packet containing artesunate tablets, and a second packet containing berberine tablets. An individual user begins the method in accordance with the present invention by administering the first loading dose of the artemether. The artemether is delivered to the individual as an injection, via IV or IM mode of administration. The second and third compositions are delivered as previously described in Examples 2-4, depending on the weight and age of the individual user.

As described in the above examples, the components of the present invention can be supplied in a kit to aid in delivery and use of the method in accordance with the present for treatments in areas where distribution channels and/or medical communities are not easily accessible or established. In accordance with the present invention, the kit may include a secure delivery package in the form of a self sealing blister pack, zip lock packs, standup pouches, foil pouches which include the single use spray bottle and one day, two day, three day, four day, five plus day packs of the artesunate and berberine. If injectable artemether is used, needles, syringes, injection bottles, or pre-loaded syringes with capped needles may be included. The kit preferably contains directions and/or any other instructions for the proper use and storage of the components of the kit.

Use of modified improved Artemisinin-based Combination Therapy incorporating delivery of an Artemisinin derivative using a sublingual, spray-based delivery route of administration

Two hundred eighty eight (288) evaluable subjects were initially randomized into two groups: Group A, receiving Standard of Care plus TriAct therapy, and Group B, receiving Standard of Care only. The Standard of Care protocol includes bed rest and liquids. The subjects were analyzed based on the presenting symptoms using the World Health Organization (WHO) classification (Dengue without warning signs (IA/IB), Dengue with warning signs (IIA/IIB) and severe dengue (IIIA/IIIB).

All study participants were confirmed cases of Dengue using NS1 antigen, as surrogate for viral load and all the clinical and laboratory parameters were compared in the two groups showing in Group A: 1) Faster elimination of viral antigen in the serum which is the culprit in the further progression of the disease into severe form like plasma leakage and severe bleeding; 2) Rapid resolution of the clinical signs and symptoms of Dengue; 3) Faster normalization of the laboratory parameters such as white blood cells (WBC) and platelet count. None of these observations had been noted in the control group (Group B).

At the completion of the clinical study, statistics validated that the modified improved Artemisinin-based Combination Therapy treatment protocol was proven safe and effective, with no deaths or adverse reactions. The modified improved Artemisinin-based Combination Therapy treatment shortened the disease process, decreased viral antigen (NS1), risk reduction of bleeding, faster normalization of the hematological picture, early resolution of the clinical symptoms and enhancement of the immune system allowing early production of neutralizing antibodies,

### Materials and Methods:

The study carried out was a prospective, open-labeled randomized, controlled uni-center study, which enrolled 300 subjects stratified into two treatment arms: Group A, WHO Standard of Care plus TriAct therapy, and Group B, WHO Standard of Care only. Subjects included individuals of ages 5 years to 65 years old admitted as cases of dengue, screened by careful history and physical examination complying with the inclusion criteria. All of subjects were positive to Dengue NS1 and/or Dengue IgM/IgG. All subjects were informed and voluntarily signed consent/assent forms. The WHO/DOH Revised Dengue Guidelines was used in the conduct of the study. The subjects in Groups A and B were further stratified into Dengue without Warning Signs (Dengue Fever/DHF I), Dengue with Warning signs (DHF II) and Severe Dengue (DHF III/IV). The research subjects were monitored at least 30 days after the last dose of the test particle.

The study used the test drug combination of Artemether (60 mg solution in a Sublingual Nano Spray) as a loading dose, Artesunate 50 mg/tablet and Berberine 400 mg/tablet. Directions and dosages for age group and weight were performed based on Table VI.

TABLE VI
Dose Schedule

| Adult | Adult/Adolescent | Children |
|---|---|---|
| (>75 Kg ox | (30-75 Kg ox 66- | (15-30 Kg ox |
| >165 lbs. ) | 165 lbs. ) | 33-66 lbs. ) |
| Artemether | Artemether | Artemether |
| 2 tablets packet | 1 tablet packet | 1 tablet packet |
| A | A | A |
| 2 tablets packet | tablet packet | 1 tablet packet |
| B | B | B |
| Every 8 hours | Every 8 hours | Every 8 hours |
| for 2 days (3 | for 2 days (3 | for 2 days (3 |
| doses/day) | doses/day) | doses/day) |

Packet A = artesunate
Packet B = berberine

All subjects had daily CBC with Actual Platelet count evaluations. NS1 determinations were done daily until the results became negative. Dengue IgM/IgG was also determined daily until the patients discharged. Other laboratory examinations like Prothrombin Time (PT), Partial Prothrombin Time (PTT), blood test for aspartate aminotransferase (AST), alanine aminotransferase (ALT), and Urinalysis were also done upon enrollment and were repeated at least on Day 7 of illness. Chest X-Ray and abdominal ultrasound were done on all subjects on the first 24 hours of defervescense. Chest X-ray was repeated at least 72 hours after. Abdominal Ultrasound was repeated for those subjects with abnormal findings.

Daily assessments of all the subjects were performed. Statistical analyses were performed using independent Samples T-test and Chi Square test for independence using SPSS 11.5.

Results: Of the 300 confirmed subjects enrolled, 288 qualified for evaluation and the rest were not able to complete the data set required. Daily evaluations of the clinical and laboratory parameters were done. Differences in Group A vs Group B were shown for the following parameters: days of illness, p=0.038, Cramer's V=0.201; days patient had a fever, p<0.001, Cramer's V=0.516; days with headache, p<0.001, Cramer's V=0.389; days with body malaise, p<0.001, Cramer's V=0.389; days with joint pain, p=0.012, Cramer's V=0.210, days with nausea/vomiting, p=0.001, Cramer's V=0.265; days with abdominal pain, p<0.001, Cramer's V=0.540; days with abdominal tenderness, p<0.001, Cramer's V=0.586; days to normalize white blood count, p<0.001, Cramer's V=0.545; days to normalize hemoglobin, p<0.001, Cramer's V=0.311; days to normalize hematocrit, p<0.001, Cramer's V=0.360; days to normalize absolute platelet count, p<0,001, Cramer's V=0.610; days before becoming negative for NS1, p<0.001, Cramer's V=0.589; days before becoming positive for IgM, p<0.001, Cramer's V=0.343; days before becoming positive for IgG, p<0.001, Cramer's V=0.293; NS1 based on day of illness, p<0.001, Cramer's V=0.638.

For the clinical parameters, the study drug group, Group A showed faster recovery and resolution of the clinical signs and symptoms

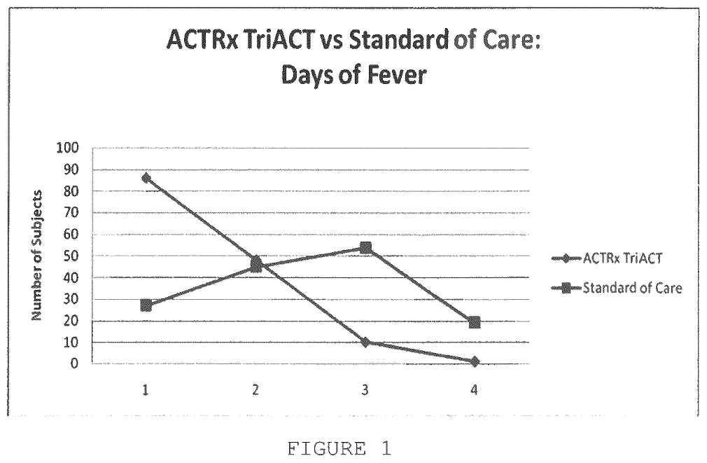

Defervesence was attained one day earlier for group A than for the control group, Group B (p<0.001), see FIG. 1.

All subjects in Group A had resolution of the body malaise and retro-orbital pain within a 3-day period compared to Group B, where there was persistence of the aforementioned symptoms up to day 5 (p<0.001 and p=0.511, respectively).

Majority (98.6%) of the subjects in group A had resolution of joint pains within the 2-day period, compared to the Group B (p=0.012).

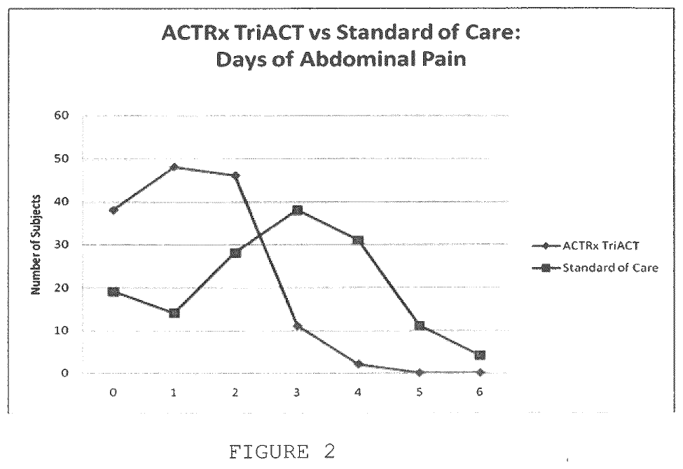

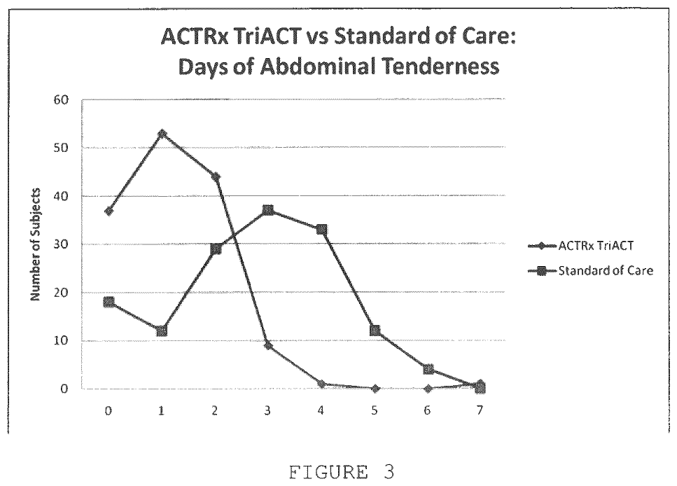

The most remarkable observation between the 2 groups was seen in the difference in abdominal pain and abdominal tenderness, including nausea and vomiting, wherein almost all of the subjects of Group A (98.6%, 98.6% and 99.3%, respectively) had resolution by day 3 as compared to the Group B (p<0.001, p<0.001 and p=0.001, respectively) by day 6 and 4, see FIGS. 2 and 3.

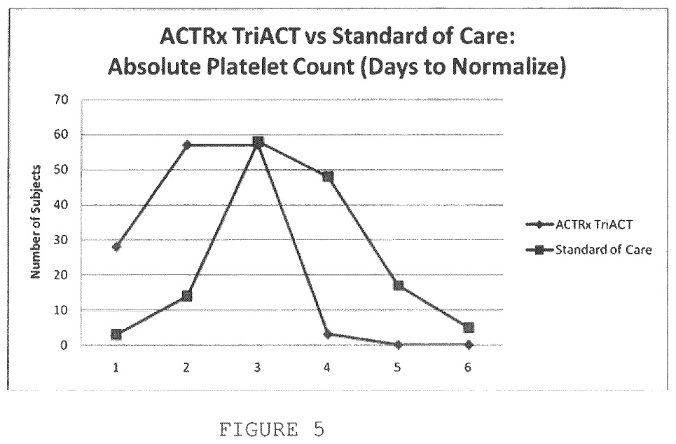

In terms of the laboratory parameters like WBC and Absolute Platelet Count, all subjects in group A showed normalization within 4 days of treatment versus the control group which took 7 days (p<0.001) and 6 days (p<0.001) see FIG. 5, respectively, to attain normalization.

The hemoglobin and hematocrit remained stable Group A subjects during the entire course of the study as compared to the control group, where 2.7% (p<0.001) and 4.1% (p<0.001), respectively, remained unstable up to day 5. It is expected, however, that there will be no significant difference in terms of the percentage increase in the hematocrit since the standard of care was applied to both treatment groups.

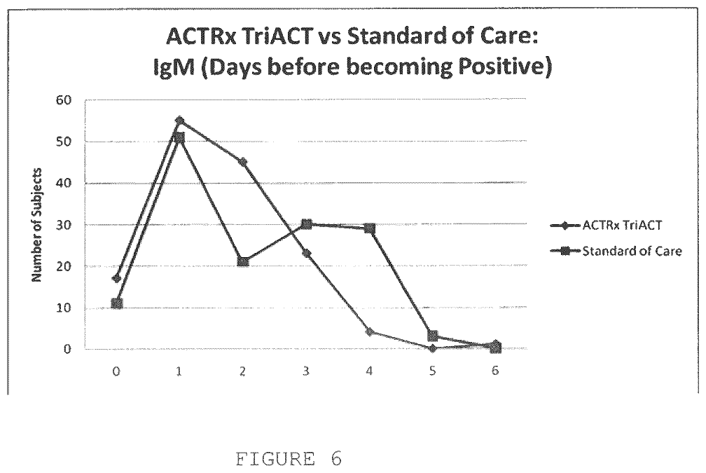

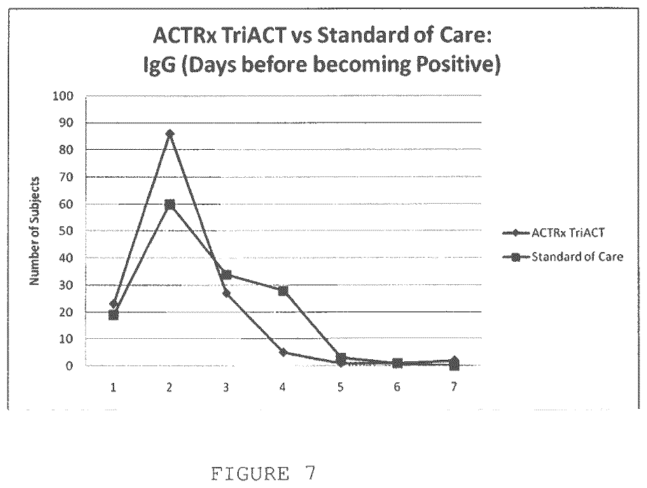

Studies further show that there was an early specific anti-body production in group A (IgM 96.5% by day 3) compared to the control group, group B (IgM 77.9%) with a p value of <0.001, see FIG. 6. The early specific anti-body production is believed to help in the rapid elimination of the virus in the body decreasing the chance of antigen-antibody complexes to form thereby preventing the eventual plasma leakage. FIG. 7 illustrates days before coming positive with IgG for group A and for the control group, Group B

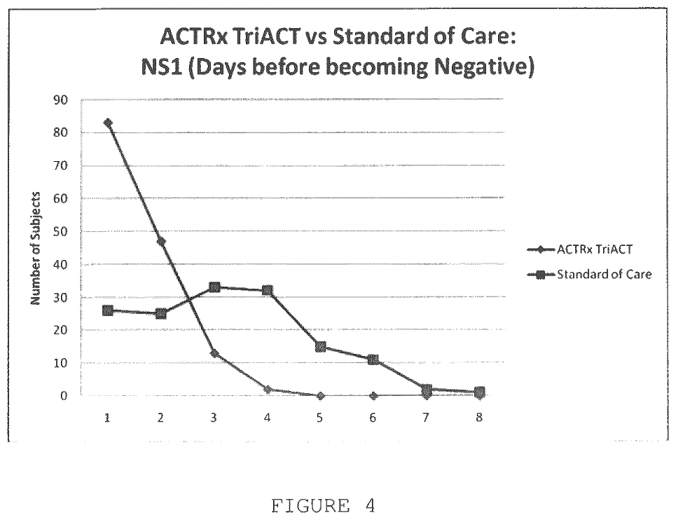

The disintegration of NS1 antigen was observed to be faster in Group A, with all of the subjects being negative on day 4. Such finding was different for the control group, Group B, which took up to day 8 for some, but not all of the subjects to become negative (p<0.001), see FIG. 4.

It is postulated that NS1 antigen may be necessary for dengue viral replication hence early disintegration of NS1 antigen means that viral replication has stopped which is probably the mechanism of action of TriAct therapy.

The reduction of the number of subjects exhibiting elevated Serum Glutamic Oxaloacetic Transaminase (SGOT) means there is no further invasion of the liver cells in the Group A (38/84) when compared to control group, Group B, (51/84) (p value of =0.044). However both groups exhibited elevated Serum Pyruvic Transaminase (SGPT). There was continuous elevation of SGPT in 62/84 (73.8%) in Group A, as compared to the Group B, 51/84 (60.7%) (p=0.071).

The present study validates the use of TriACt therapy as an effective combination therapy for effectively treating Dengue. The study indicates that TriACt therapy was safe as there were no adverse reactions recorded in the treatment group. Use of the TriACt therapy was found to offer several unexpected benefits as a treatment program. The TriAct therapy eliminated the virus fifty percent (50%) faster as documented by the disappearance of NS1. The number of days of illness was significantly reduced by at least fifty percent (50%) in those patients treated with the TriAct therapy, something that was not anticipated. The TriAct therapy reduced the number of days of incapacitation in those patients treated with the TriAct therapy by at least fifty percent (50%). The TriAct therapy group recovered faster from fever. There was a significant decrease in abdominal pain and abdominal tenderness in the treated group, a finding that surprised investigators. The treatment allowed complete recovery from retro-orbital pain. Using the TriAct therapy, rapid normalization of white blood cells and platelet counts resulted. The cessation of bleeding was a critical finding in this study. Significant reduction of body malaise and joint pains was also shown for individuals within the TriAct therapy group. Early cessation of nausea/vomiting was attained in the TriAct therapy group.

All patents and publications mentioned in this

specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of treating Dengue comprising the steps of:
administering to an individual a first composition comprising a sublingually delivered, single dosage of artemether or its pharmaceutically acceptable salt in the range of 50 to 80 mg per dosage, said single dosage remaining under said individual's tongue for at least 30 seconds;
after a period of 30 minutes post administration of said single dosage of artemether or its pharmaceutically acceptable salt, administering to said individual a second composition two or three times a day for at least two days, said second composition comprising one or more doses of artesunate or its pharmaceutically acceptable salt in the range of 40 to 100 mg per each individual dosage;
after a period of 30 minutes from administrating said single dosage of artemether or its pharmaceutically acceptable salt, administering to said individual a third composition two or three times a day for at least two days, said third composition comprising one or more doses of berberine, or its pharmaceutically acceptable salt in the range of 200 to 400 mg per each individual dosage.

2. The method according to claim 2, wherein said second and said third compositions are delivered via an oral route.

3. The method according to claim 1, wherein said second and said third compositions are in the form of a pill or capsule.

4. The method according to claim 1, wherein said step of administering said artemether includes using a single dosage spray.

5. The method according to claim 1, further including the steps of administering additional doses of said second and said third compositions for a predetermined time period.

6. The method according to claim 1, wherein said artemether is delivered as a spray.

7. The method according to claim 6, wherein said spray includes a mucosal absorption enhancer.

8. A method of treating Dengue comprising the steps of:
on day one, sublingually administering to an individual a single dosage of a therapeutically effective amount of artemether or its pharmaceutically acceptable salt, delivered as a spray;
waiting at least 30 minutes after administration of said artemether or its pharmaceutically acceptable salt, administering to said individual, as an initial dosage, a therapeutically effective amount of artesunate or its pharmaceutically acceptable salt and a therapeutically effective amount of berberine, or its pharmaceutically acceptable salt,
administering one or two additional dosages of said therapeutically effective amount of artesunate or its pharmaceutically acceptable salt and said therapeutically effective amount of berberine, or its pharmaceutically acceptable salt; and
on day two, administering two or three dosages of said therapeutically effective amount of artesunate or its pharmaceutically acceptable salt and said therapeutically effective amount of berberine, or its pharmaceutically acceptable salt.

9. The method according to claim 8, further including the steps of administering two or three dosages of said therapeutically effective amount of artesunate or its pharmaceutically acceptable salt and said therapeutically effective amount of berberine, or its pharmaceutically acceptable salt for a period of two additional days.

10. The method according to claim 8, wherein said spray includes a mucosal absorption enhancer.

11. The method according to claim 8, wherein a delivery dose of said artemether is between 50 and 80 mg per dosage.

12. The method according to claim 8, wherein said therapeutically effective amount of artemether or its pharmaceutically acceptable salt is administered as a single dosage spray of between 50 and 80 mg.

13. The method according to claim 11, wherein said single dosage spray is 80 mg.

14. The method according to claim 8, wherein an amount of said artesunate delivered per dose is 20 mg to 250 mg.

15. The method according to claim 14, wherein said artesunate is delivered three times per day, each delivery containing a dose of between 20 mg to 250 mg.

16. The method according to claim 8, wherein an amount of said berberine delivered per dose is between 200 mg and 600 mg.

17. The method according to claim 8 wherein said amount of said berberine delivered per dose is at least 400 mg.

18. A kit for treating a microbial disease comprising:
a first composition comprising a therapeutically effective amount of artemether or its pharmaceutically acceptable salt;
a second composition comprising a therapeutically effective amount of artesunate or its pharmaceutically acceptable salt; and
a third composition comprising a therapeutically effective amount of berberine, or its pharmaceutically acceptable derivatives.

19. The kit for treating a microbial disease according to claim 18, wherein said artemether is preloaded to a syringe or is in the form of a single dosage spray.

20. The kit for treating a microbial disease according to claim 18, wherein said second and said third compositions are in pre-packed dosages.
